(19) United States
(12) Patent Application Publication
(10) Pub. No.: US 2017/0266147 A1
(43) Pub. Date: Sep. 21, 2017

(54) METHODS OF INCREASING LATENCY OF ANESTHETIC INDUCTION USING KETONE SUPPLEMENTATION

(71) Applicants: Csilla Ari D'Agostino, Tampa, FL (US); Dominic Paul D'Agostino, Tampa, FL (US)

(72) Inventors: Csilla Ari D'Agostino, Tampa, FL (US); Dominic Paul D'Agostino, Tampa, FL (US)

(21) Appl. No.: 15/463,887

(22) Filed: Mar. 20, 2017

Related U.S. Application Data
(60) Provisional application No. 62/310,302, filed on Mar. 18, 2016.

(51) Int. Cl. A61K 31/225; A61K 31/20; A61K 38/02; A23L 33/10; A23L 33/00; A23L 33/115; A23L 33/17; A61K 31/19; A61K 31/70

(52) U.S. Cl. CPC A61K 31/225; A61K 31/19; A61K 31/20; A61K 38/02; A61K 31/70; A23L 33/40; A23L 33/115; A23L 33/17; A23L 33/10; A23V 2002/00

(57) ABSTRACT

Provided are methods for delaying the onset of anesthesia in a subject.

### CROSS-REFERENCE TO RELATED APPLICATIONS

This application claims priority to U.S. Provisional Application No. 62/310,302 filed on Mar. 18, 2016, which is incorporated fully herein by reference.

### TECHNICAL FIELD

The present disclosure relates to compositions for increasing latency to anesthetic induction in a subject.

### BACKGROUND

The primary fuel source for the human body is glucose, a sugar that is metabolized in the liver to yield acetyl-CoA, which drives the citric acid cycle to produce ATP. In the absence of glucose, the body switches to metabolizing fatty acids for fuel, generating ketone bodies that can be transported out of the liver to other tissues in the body. The ketone bodies are then converted back to acetyl-CoA in the mitochondria of the glucose-deprived tissue, allowing the citric acid cycle to continue generating ATP when glucose supplies are low. Importantly, ketone bodies are able to cross the blood-brain-barrier and provide fuel to the brain when glucose is in short supply. An increase in ketone bodies, called ketosis, ensures that cells will continue to function properly in the absence of glucose, however an excess of ketone bodies can lower the pH of the blood and cause ketoacidosis, which can damage vital organs.

However, when managed appropriately, long-term ketosis has proven to be beneficial in treating a number of medical conditions. Long-term ketosis has been shown to improve the symptoms of patients with Alzheimer's disease, glucose transporter type 1 (GLUT1)-deficiency syndrome, cancer, and epilepsy. Elevated blood ketone body levels have also been associated with neuroprotection.

Given the health benefits associated with ketosis, there remains a need in the art for identifying effective techniques for inducing ketosis in a subject to achieve a desired result. Provided herein are such methods.

### SUMMARY

The present invention is directed to a method of delaying the onset of anesthetic induction, comprising administering a composition comprising at least one ketone supplements to a subject, wherein ketosis is induced in the subject. The ketone supplements may be at least one of a ketone salt, a ketone ester, or a ketone body precursor, or any combination thereof. The ketone supplement may be 1,3 butanediol-acetoacetate diester. The ketone supplement may be Na⁺/K⁺ βHB mineral salt. The subject may be a human.

The present invention is also directed to a method of delaying the onset of anesthetic induction, comprising administering a ketogenic diet to a subject, wherein ketosis is induced in the subject. The ketogenic diet may comprise fats, proteins, and carbohydrates. The ketogenic diet may be administered with a composition comprising a ketone supplement.

### BRIEF DESCRIPTION OF THE DRAWINGS

FIG. 1 shows a bar graph illustrating the effects of ketosis on the time required for anesthetic induction in Sprague-Dawley rats.

FIG. 2 shows a bar graph illustrating the effects of ketosis on blood levels of β-hydroxybutyrate after anesthetic induction in Sprague-Dawley rats.

FIG. 3 shows a bar graph illustrating the effects of a standard diet with ketone ester on the time required for anesthetic induction in a glucose transporter type 1-deficiency (G1D) syndrome mouse model.

FIG. 4 shows a bar graph illustrating the effects of ketosis on the time required for anesthetic induction in G1D mice.

FIG. 5 shows a bar graph illustrating the time until anesthetic induction after ketosis, normalized to body weight in G1D mice.

FIG. 6 shows a bar graph illustrating the effects of ketosis on blood levels of β-hydroxybutyrate in G1D mice.

FIG. 7 shows a line graph indicating a positive correlation between the time until anesthetic induction and blood levels of β-hydroxybutyrate in G1D mice.

### DETAILED DESCRIPTION

The present disclosure is predicated, at least in part, on the discovery that establishing ketosis in a subject can delay the onset of anesthesia. Ketosis can be achieved by administering a composition comprising a ketogenic supplement, (e.g., a ketone, salt, a ketone ester, a ketone body precursor, a derivative of a ketone body, or combinations thereof), or by introducing a ketogenic diet, such as a diet that is high in fats and low in carbohydrates. Delaying the onset of anesthesia allows surgeons and anesthesiologists more flexibility to communicate important instructions to a patient prior to loss of consciousness for a medical procedure; ketosis may also reverse the effects of anesthesia, preventing accidental deaths during surgery and allowing for a faster recovery. Ketosis may additionally help deep sea divers delay the onset of nitrogen narcosis, a drowsy state induced by breathing air under high pressure, and may also provide resistance to harmful gases, such as carbon monoxide, volcanic gases, or chemical weapons/warfare. The present disclosure describes a method to delay the onset of anesthesia in a subject, comprising administering to the subject a composition comprising one or more ketone supplements.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise indicated herein, and each separate value is incorporated into the specification as if it were individually recited herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The singular forms “a,” “and,” and “the” include plural references unless the context clearly dictates otherwise. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “administration” or “administering” is used throughout the specification to describe the process by which the disclosed compositions may be delivered to a subject. Administration will often depend upon the amount of composition administered, the number of doses, and duration of treatment. Multiple doses of the composition may be administered. The frequency of administration of the composition can vary depending on any of a variety of factors, such as the level of ketone bodies in the blood, and the like. The duration of administration of the composition, e.g., the period of time over which the composition is administered, can vary, depending on any of a variety of factors, including patient response, etc.

The amount of the composition administered can vary according to factors such as the degree of susceptibility of the individual, the age, sex, and weight of the individual, idiosyncratic responses of the individual, the dosimetry, and the like. Detectably effective amounts of the composition of the present disclosure can also vary according to instrument and film-related factors. Optimization of such factors is well within the level of skill in the art.

“Anesthetic induction” as used herein, refers to the administration of an anesthetic drug or combination of anesthetic drugs to achieve a state of general anesthesia in a subject. General anesthesia is characterized by, for example, suppressed metabolic functions (e.g., suppressed heart rate, suppressed breathing rate), muscle paralysis, amnesia, analgesia, sedation, and/or loss of consciousness. An anesthetic drug may be in the form of a liquid, and administered by, for example, intravenous or subcutaneous routes. An anesthetic drug may be in the form of a gas, and administered by, for example, inhalation of the gas by a subject.

The terms “beta-hydroxybutyrate,” “βHB,” or “BHB,” as used interchangeably herein, refer to a carboxylic acid having the general formula CH₃CH₂OHCH₂COOH. βHB is a ketone body which may be utilized by the body as a fuel source during instances of low glucose levels.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

“Derivative” refers to a compound or portion of a compound that is derived from or is theoretically derivable from a parent compound.

The term “hydroxyl group” is represented by the formula —OH.

The term “alkoxy group” is represented by the formula —OR, where R can be an alkyl group, including a lower alkyl group, optionally substituted with an alkenyl, alkynyl, aryl, aralkyl, cycloalkyl, halogenated alkyl, or heterocycloalkyl group, as defined below.

The term “ester” as used herein is represented by the formula —OC(O)R, where R can be an alkyl, alkenyl, alkynyl, aryl, aralkyl, cycloalkyl, halogenated alkyl, or heterocycloalkyl group, as defined below.

The term “alkyl group” is defined as a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 10 carbon atoms.

The term “alkenyl group” is defined as a hydrocarbon group of 2 to 24 carbon atoms and structural formula containing at least one carbon-carbon double bond.

The term “alkynyl group” is defined as a hydrocarbon group of 2 to 24 carbon atoms and a structural formula containing at least one carbon-carbon triple bond.

The term “halogenated alkyl group” is defined as an alkyl group as defined above with one or more hydrogen atoms present on these groups substituted with a halogen (F, Cl, Br, I).

The term “cycloalkyl group” is defined as a non-aromatic carbon-based ring composed of at least three carbon atoms. Examples of cycloalkyl groups include, but are not limited to, cyclopropyl, cyclobutyl, cyclopentyl, cyclohexyl, etc.

The term “heterocycloalkyl group” is a cycloalkyl group as defined above where at least one of the carbon atoms of the ring is substituted with a heteroatom such as, but not limited to, nitrogen, oxygen, sulfur, or phosphorous.

The term “aliphatic group” is defined as including alkyl, alkenyl, alkynyl, halogenated alkyl and cycloalkyl groups as defined above. A “lower aliphatic group” is an aliphatic group that contains from 1 to 10 carbon atoms.

The term “aryl group” is defined as any carbon-based aromatic group including, but not limited to, benzene, naphthalene, etc.

The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorous. The aryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl group can be unsubstituted.

The term “aralkyl” is defined as an aryl group having an alkyl group, as defined above, attached to the aryl group. An example of an aralkyl group is a benzyl group.

“Esterification” refers to the reaction of an alcohol with a carboxylic acid or a carboxylic acid derivative to give an ester.

“Transesterification” refers to the reaction of an ester with an alcohol to form a new ester compound.

“Ketone” or “ketone body” are used interchangeably herein and refer to a compound or species which is β-hydroxybutyrate (βHB), acetoacetate (AcAc), acetone, or a combination thereof. A ketone body may be derived from a ketone body precursor, that is, a compound or species which is a precursor to a ketone body and which may be converted or metabolized to a ketone body in a subject. Any suitable ketone body precursor may be used.

“Ketone body ester” or “ketone ester” as used herein, refer to an ester of a ketone body, ketone body precursor, or a derivative thereof. Any suitable ketone ester known in the art may be used. For example, the ketone ester may be 1,3-butanediol acetoacetate diester.

“Ketone body salt” or “ketone salt” as used herein, is a salt of a ketone body, a ketone body precursor, or a derivative thereof. The ketone body salt may be combined with a monovalent cation, divalent cation, or alkaline amino acid. Any suitable ketone salt known in the art may be used. For example, the ketone salt may be a βHB salt.

The terms “ketogenic state” or “ketosis” as used interchangeably herein, refer to a subject having blood ketone body levels within the range of about 0.5 mmol/L to about 10 mmol/L. Levels above 10 mmol/L are associated with ketoacidosis, a symptom of type-1 diabetes. Ketosis may be achieved in a subject by administering a ketogenic diet or a composition comprising one or more ketone supplements.

“Keto-adaptation” as used herein refers to prolonged nutritional ketosis (>1 week) to achieve a sustained non-pathological ketosis.

A “ketone supplement” or “ketogenic compound” as used interchangeably herein, refers to a composition comprising a compound capable of elevating ketone body concentrations in a subject. The ketone supplement may be derived from, for example, a ketone body precursor, a ketone ester, a ketone salt, or a combination thereof.

“Ketogenic diet” as used herein refers to a diet that causes a metabolic switch from burning glucose for energy to burning fats for energy. Nutritional ketosis/ketogenic state may be achieved through calorie restriction, fasting, prolonged exercise, and/or a ketogenic diet that is high in fat and restricted in carbohydrates (e.g. sugars).

The term “medium chain triglycerides” (MCT) as used herein refers to molecules having a glycerol backbone attached to three medium chain fatty acids. Medium chain fatty acids range from 6 to 12 carbon atoms in length.

A “therapeutically effective amount,” or “effective dosage” or “effective amount” as used interchangeably herein unless otherwise defined, means a dosage of a drug effective for periods of time necessary, to achieve the desired therapeutic result. An effective dosage may be determined by a person skilled in the art and may vary according to factors such as the disease state, age, sex, and weight of the individual, and the ability of the drug to elicit a desired response in the individual. This term as used herein may also refer to an amount effective at bringing about a desired in vivo effect in an animal, vertebrate, mammal, or human, such as increasing the time until anesthetic induction.

A therapeutically effective amount may be administered in one or more administrations (e.g., the composition may be given as a preventative treatment or therapeutically at any stage of disease progression, before or after symptoms, and the like), applications or dosages and is not intended to be limited to a particular formulation, combination or administration route. It is within the scope of the present disclosure that the ketogenic diet or ketone supplement may be administered at various times during the course of treatment of the subject. The times of administration and dosages used will depend on several factors, such as the goal of treatment (e.g., treating v. preventing), condition of the subject, etc., and can be readily determined by one skilled in the art.

A “pharmaceutically acceptable excipient,” “pharmaceutically acceptable diluent,” “pharmaceutically acceptable carrier,” or “pharmaceutically acceptable adjuvant” as used herein means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use, such as those promulgated by the United States Food and Drug Administration.

“Subject” and “patient” as used herein interchangeably refers to any vertebrate, including, but not limited to, mammals (e.g., cow, pig, camel, llama, horse, goat, rabbit, sheep, hamsters, guinea pig, cat, dog, rat, and mouse, a non-human primate (for example, a monkey, such as a cynomologus or rhesus monkey, chimpanzee, etc.) and a human). The subject may be a human or a non-human. The subject or patient may be undergoing other forms of treatment.

### 2. ESTABLISHING KETOSIS IN A SUBJECT

The disclosure provides for methods of delaying anesthetic onset by establishing ketosis in a subject. Nutritional ketosis is distinguished from diabetic or alcoholic ketoacidosis. Diabetic ketoacidosis is associated with, for example, the absence of insulin, blood ketone levels in excess of 10 mmol/L, metabolic derangement, and electrolyte imbalance. Alcoholic ketoacidosis is associated with an excessive accumulation of blood ketone body levels and a drop in blood pH.

The disclosure provides methods for delaying the onset of anesthesia in a subject. The method may comprise administering to the subject a composition comprising one or more ketone supplements. The ketone supplement may be any compound capable of elevating ketone body concentrations in a subject. For example, the ketone supplement may elevate expression of βHB, acetoacetate, acetone, or a combination thereof, following administration to the subject. The ketone supplement may be a ketone body precursor or derivative thereof. Any suitable ketone body precursor which will be metabolized into a ketone body upon administration to the subject may be used. For example, the ketone supplement may comprise any one or more of 1,3-butanediol, acetoacetate, or βHB moieties or derivatives thereof, including esters and salts thereof. For example, the ketone supplement may be 1,3-butanediol, ethyl acetoacetate, or ethyl βHB.

The ketone supplement may be a ketone ester. Any suitable ketone ester may be used in the disclosed composition. The ketone ester may be a monoester or a diester. The ketone ester may be a glycerol monoester or diester. In an embodiment, the monoester may be esterified at the 1 position. In another embodiment, the diester may be esterified at the 1 and 3 positions. In an embodiment, the ketone ester may comprise a monoester of butane-1,3-diol with D-3-hydroxybutyrate or L-3-hydroxybutyrate, for example 3-hydroxybutyl-L,D-β-hydroxybutyrate, and a monoester and a diester of glycerol with D-3-hydroxybutyrate or L-3-hydroxybutyrate. The ester may be in an enantiomerically enriched form. Compounds which provide a ketone body in situ include esters of βHB and oligomers of βHB, such as, for example, esters derived from alcohols and compounds containing one or more free hydroxyl groups. Suitable alcohols include butanediol (e.g., butane-1,3-diol), altrose, arabinose, dextrose, erythrose, fructose, galactose, glucose, glycerol, gulose, idose, lactose, lyxose, mannose, ribitol, ribose, ribulose, sucrose, talose, threose, xylitol, and xylose.

The ketone supplement may be a ketone salt. Any suitable ketone salt may be used in the disclosed composition. For example, the ketone salt may be βHB mineral salt. The βHB may be the D- or L-enantiomer, also described as the R or S configuration. In another embodiment, the βHB may be monomeric. The ketone salt may comprise, for example, sodium (Na⁺) βHB, potassium (K⁺) βHB, calcium (Ca⁺) βHB, lithium (Li⁺) βHB, magnesium (Mg²⁺) βHB, or any other feasible non-toxic mineral salts of βHB. Organic salts of βHB include salts of organic bases such as arginine βHB, lysine βHB, histidine, βHB ornithine βHB, creatine βHB, agmatine βHB, and citrulline βHB. The ketone salt may be a combination of any of the βHB salts. For example, the ketone salt may be sodium βHB and arginine βHB, or βHB sodium salt and βHB potassium salt.

In an embodiment, the ketone salt may be comprised in a solution. Preferably, the βHB mineral salt may be from 5% to 60% of a solution. For example, the βHB mineral salt may be 5%, may be 6%, may be 7%, may be 8%, may be 9%, may be 10%, may be 11%, may be 12%, may be 13%, may be 14%, may be 15%, may be 16%, may be 17%, may be 18%, may be 19%, may be 20%, may be 21%, may be 22%, may be 23%, may be 24%, may be 25%, may be 26%, may be 27%, may be 28%, may be 29%, may be 30%, may be 31%, may be 32%, may be 33%, may be 34%, may be 35%, may be 36%, may be 37%, may be 38%, may be 39%, may be 40%, may be 41%, may be 42%, may be 43%, may be 44%, may be 45%, may be 46%, may be 47%, may be 48%, may be 49%, may be 50%, may be 51%, may be 52%, may be 53%, may be 54%, may be 55%, may be 56%, may be 57%, may be 58%, may be 59%, or may be 60%. Preferably, the βHB mineral salt is about 50% of a solution.

In an embodiment, the βHB mineral salt is comprised of about 375 mg/g of pure βHB and about 125 mg/g of Na⁺/K⁺. The dose of the ketone salt may be from about 1000 mg to about 25,000 mg of βHB, depending on the weight of the subject. For example, the dose of the βHB mineral salt may be from about 1100 mg to about 24,000 mg, about 1200 mg to about 23,000 mg, about 1300 mg to about 22,000 mg, about 1400 mg to about 21,000 mg, about 1500 mg to about 20,000 mg, about 1600 mg to about 19,000 mg, about 1700 mg to about 18,000 mg, about 1800 mg to about 17,000 mg, about 1900 mg to about 16,000 mg, about 2000 mg to about 15,000 mg, about 2100 mg to about 14,000 mg, about 2200 mg to about 13,000 mg, about 2300 mg to about 12,000 mg, about 2400 mg to about 11,000 mg, about 2500 mg to about 10,000 mg, about 2600 mg to about 9000 mg, about 2700 mg to about 8000 mg, about 2800 mg to about 7000 mg, about 2900 mg to about 6000 mg, about 3000 mg to about 5000 mg, or about 3100 mg to about 4000 mg. For example, the dose may be about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, about 20,000 mg, about 21,000 mg, about 22,000 mg, about 23,000, mg, about 24,000 mg, or about 25,000 mg.

The composition may additionally comprise at least one medium chain fatty acid or ester thereof. For example, the composition may additionally comprise at least one medium chain triglyceride (MCT). For example, the composition may comprise a ketone salt with MCT, a ketone ester with MCT, a ketone ester and a ketone salt with MCT, or combinations thereof. Sources of the medium chain fatty acid, or an ester thereof, include coconut oil, coconut milk powder, fractionated coconut oil, palm oil, palm kernel oil, caprylic acid, isolated medium chain fatty acids such as isolated hexanoic acid, isolated octanoic acid, isolated decanoic acid, medium chain triglycerides either purified or in natural form such as coconut oil, and ester derivatives of the medium chain fatty acids ethoxyiated triglyceride, enone triglyceride derivatives, aldehyde triglyceride derivatives, monoglyceride derivatives, diglyceride derivatives, and triglyceride derivatives, and salts of the medium chain triglycerides. Ester derivatives optionally include alkyl ester derivatives, such as methyl, ethyl, propyl, butyl, hexyl, etc. Derivatives may be prepared by any process known in the art, such as direct esterification, rearrangement, fractionation, transesterification, or the like. The composition may comprise a ketone salt and a MCT mixed at an approximate 1:1 ratio. The composition may comprise a ketone ester and a MCT mixed at an approximate 1:1 ratio. The composition may comprise a ketone precursor and a MCT mixed at an approximate 1:1 ratio. The composition may comprise MCT oil. Suitably, the MCT oil may comprise 65% caprylic triglyceride.

The disclosed composition may comprise any combination of one or more ketone supplements. For example, the disclosed composition may comprise a combination of any one or more of a ketone ester, a ketone salt, a ketone body precursor, a medium chain fatty acid, or combinations thereof. The composition may comprise at least one ketone salt and at least one ketone ester. For example, the composition may comprise sodium/potassium βHB mineral salt and 1,3-butanediol acetoacetate diester. The composition may comprise at least one ketone salt and at least one medium chain fatty acid. For example, the composition may comprise sodium/potassium βHB mineral salt and a MCT. The composition may comprise at least one ketone ester and at least one medium chain fatty acid. For example, the composition may comprise 1,3 butanediol acetoacetate diester and a MCT. The above combinations are intended strictly to provide examples and are in no way limiting to other combinations that may be used.

The composition may additionally comprise other nutritional substrates. For example, the composition may additionally comprise free amino acids, amino acid metabolites, vitamins, minerals, electrolytes and metabolic optimizers such as NADH, soluble ubiquinol, tetrahydrobiopterin, alpha-ketoglutaric acid, carnitine, and/or alpha lipoic acid, nutritional co-factors, calcium beta-methyl-beta-hydroxybutyrate, arginine alpha-ketoglutarate, sodium R-alpha lipoic acid, thiamine, riboflavin, niacin, pyridoxine, ascorbic acid, citric acid, malic acid, sodium benzoate, potassium sorbate, acesulfame K, aspartame, xanthan gum, or a combination thereof. Non-limiting examples of nutritional co-factors include R-alpha lipoic acid, acetyl-1-carnitine, ketoisocaproate, alpha-ketoglutarate, alpha-hydroxyisocaproate, creatine, branched chain amino acids (leucine, isoleucine, valine), beta-hydroxy-beta methylbutyrate (HMB), B vitamins, vitamin C, soluble ubiquinol, and carnitine that assist in mitochondrial function.

The composition may be administered in various dosages to the subject. For example, the composition may be administered in a dosage range of 1 mg ketone supplement/kg of body weight to 100 g ketone supplement/kg body weight.

The composition may be delivered to the subject in any dose sufficient to achieve the desired therapeutic effect, e.g. a delay in anesthetic induction in the subject. For example, a therapeutically effective amount of a ketone supplement of the disclosed composition may be about 1 mg ketogenic compound/kg body weight to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg. For example, a therapeutically effective amount of a ketone supplement of the disclosed composition may be about 1.2 mg/kg, about 2.5 mg/kg, about 5 mg/kg, or about 10 mg/kg.

The composition may be administered in various ways, including, for example, orally, intragastricly, or parenterally (referring to intravenously and intra-arterially and other appropriate parenteral routes), among others. Administration may be as a single dose, or multiple doses over a period of time. In an embodiment, the composition or the ketone diet may be administering chronically, for example, between about 1 day and about 7 days), or sub-chronically (e.g., more than 7 days). For example, multiple doses may be delivered over 1 day, 3 days, 5 days, 7 days, 10 days, 14 days, or more, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more, 1 month, 2 months, 3 months, 4 months, 5 months, 6 months, 7 months, 8 months, 9 months, 10 months, 11 months, 12 months, or more.

The composition may be a solid, for example a powder, tablet, bar, confectionary product, or a granule, and intended for use as a solid oral dose form. In another embodiment, the solid composition may be mixed before use with a liquid, such as a water-based liquid (e.g., fruit drink, dairy product, milk, and yogurt), to provide a liquid drink for the user. The composition may be provided, as desired, as a liquid product in a form ready for consumption or as a concentrate or paste suitable for dilution on use. The composition may be pH adjusted with citric and/or malic acid, and artificial sweetener and flavoring can be added. The liquid product may be homogenized and pasteurized. The composition may further comprise a pharmaceutically acceptable excipient, diluent, or carrier.

The present disclosure also provides for methods of delaying anesthetic induction by administering to a subject a ketogenic diet to induce ketosis in the subject. In an embodiment, a ketogenic diet may comprise foods that are high in fat, low in carbohydrates, and provide adequate protein. The ketogenic diet may comprise from about 60.0% to about 90.0% fat. For example, the ketogenic diet may comprise about 60%, about 61%, about 62%, about 63%, about 64%, about 65%, about 66%, about 6′7%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90%. In another embodiment, the ketogenic diet comprises from about 10.0% to about 25.0% protein. For example, the ketogenic diet may comprise about 10%, about 11%, about 12%, about 13%, about 14%, about 15%, about 16%, about 17%, about 18%, about 19%, about 20%, about 21%, about 22%, about 23%, about 24%, or about 25%. In yet another embodiment, the ketogenic diet comprises from about 0.1% to about 5.0% carbohydrate. For example, the ketogenic diet may comprise about 0.1%, about 0.2%, about 0.3%, about 0.4%, about 0.5%, about 0.6%, about 0.7%, about 0.8%, about 0.9%, about 1.0%, about 2.0%, about 3.0%, about 4.0%, or about 5.0%. In a preferred embodiment, the ketogenic diet consists of 77.1% fat, 22.4% protein, and 0.5% carbohydrate.

Preferably, the ketogenic diet comprises a caloric density from about 4 Kcal/g to about 8 Kcal/g. For example, the ketogenic diet may be about 4 Kcal/g, about 5 Kcal/g, about 6 Kcal/g, about 7 Kcal/g, or about 8 Kcal/g. In a preferred embodiment, the caloric density may be about 4 Kcal/g, such as about 4.1 Kcal/g, about 4.2 Kcal/g, about 4.3 Kcal/g, about 4.4 Kcal/g, about 4.5 Kcal/g, about 4.6 Kcal/g, about 4.7 Kcal/g, about 4.8 Kcal/g, about 4.9 Kcal/g, about 5.0 Kcal/g. In a preferred embodiment, the ketogenic diet comprises a caloric density of about 4.7 Kcal/g. The ketogenic diet may also comprise a composition comprising a ketone supplement (e.g., a ketone salt, a ketone ester, a ketone body precursor, or combinations thereof).

The levels of circulating glucose and ketone bodies may be measured in a subject prior to or following administration of a ketogenic diet, or a composition comprising a ketone supplement. Circulating levels may be determined from, for example, bodily fluids (e.g. blood, serum, plasma, or urine) or breath (such as, acetone on the breath). Any suitable measuring device or kit known in the art may be used, such as the PRECISION XTRA® blood glucose and ketone monitoring kit (Abbott Laboratories, Abbott Park, Ill.). The ketone body to be measured may be β-hydroxybutyrate (or 3-hydroxybutyrate), acetoacetate, acetone, derivatives thereof, or combinations thereof.

The invention further discloses a kit, which may be used to induce ketosis in a subject. Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### 3. EXAMPLES

The foregoing may be better understood by reference to the following examples, which are presented for purposes of illustration and are not intended to limit the scope of the invention. The present invention has multiple aspects, illustrated by the following non-limiting examples.

### Example 1—Ketosis in Sprague-Dawley Rats

This example describes the effects of ketosis on the time to anesthetic induction in Sprague-Dawley rats.

Animals were kept in pairs of 2 rats under standard laboratory conditions (12 h:12 h light-dark cycle) in climate-controlled rooms at 22.0±2.0° C. Animal treatment and measuring procedures were performed in accordance with the University of South Florida Institutional Animal Care and Use Committee (IACUC) guidelines (Protocol#00001749). All efforts were made to reduce the number of animals used.

All data are presented as the mean±standard error of the mean (SEM). The effects of compositions comprising ketone supplements on latency to anesthetic induction, as well as on blood βHB levels, were compared to control and/or baseline levels. Data analysis was performed using GRAPHPAD PRISM® v6.0a. Results were considered significant when *p<0.05, **p<0.005, ***p<0.0005. Significance was determined by unpaired t-test.

Sprague-Dawley rats were fed a standard diet, a ketone diet, or a standard diet supplemented with an oral gavage of the ketone salt βHB mineral salt for 7 days. After 7 days, anesthesia was induced in an anesthesia chamber with isoflurane gas mixed with air, and the time until anesthetic induction was measured by a blinded observer.

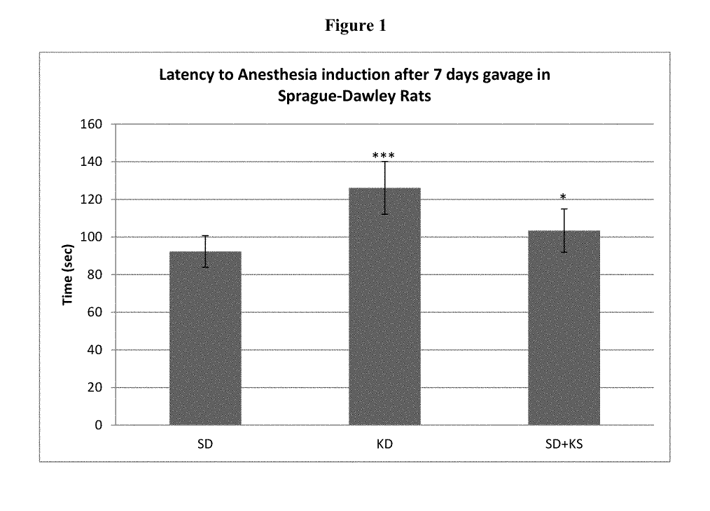

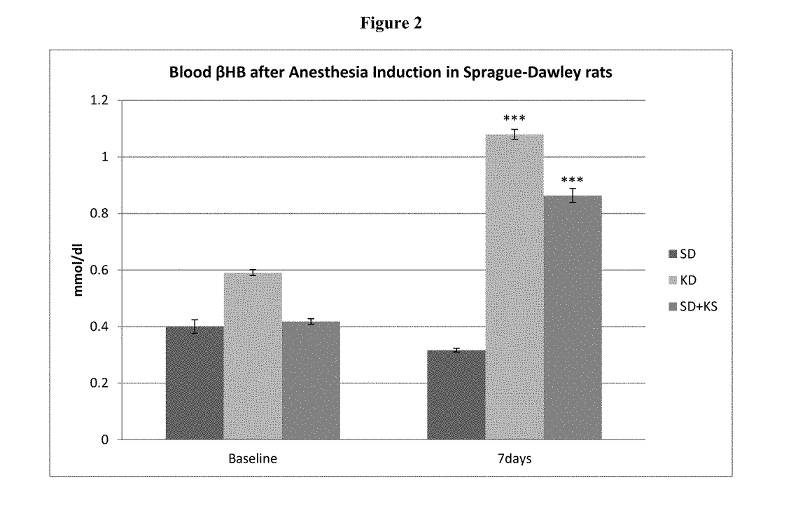

Treatment with a ketone diet, or standard diet with ketone salt, caused a significant increase in the number of seconds required before anesthetic induction (p=1.32E−6, p=0.336, relative to standard diet fed controls) (FIG. 1). Rats in these treatment groups also exhibited a significant increase in the blood levels of βHB after anesthetic induction (p=0.0001, p=2.21E−05, relative to baseline levels) (FIG. 2).

The results of this example demonstrate that ketosis induced in Sprague-Dawley rats with a ketogenic diet or a standard diet with ketone salt caused a significant increase in the number of seconds required before the onset of anesthetic induction, which correlated with a rise in blood βHB levels.

### Example 2—Ketosis in G1D Mice

This example describes the effects of ketosis on the time to anesthetic induction in a glucose transporter type 1-deficiency (G1D) syndrome mouse model of seizures.

Animals were kept in groups of 2-5 mice under standard laboratory conditions (12 h:12 h light-dark cycle) in climate-controlled rooms at 22.0±2.0° C. Animal treatment and measuring procedures were performed in accordance with the University of South Florida Institutional Animal Care and Use Committee (IACUC) guidelines (Protocol#00000457). All efforts were made to reduce the number of animals used. The rodents were put under anesthesia once a week to perform blood draws.

All data are presented as the mean±standard error of the mean (SEM). The effects of compositions comprising ketone supplements on latency to anesthetic induction, as well as on blood βHB levels, were compared to control and/or baseline levels. Data analysis was performed using GRAPHPAD PRISM v6.0a. Results were considered significant when *p<0.05, **p<0.005, ***p<0.0005. Significance was determined by unpaired t-test.

G1D mice between 3-5 months old were chronically fed a standard rodent chow diet (SD), ketogenic diet (KD) (Table 1), standard rodent chow diet mixed with 20% ketone salt (βHB mineral salt) supplementation (SD+KS), or standard diet mixed with 20% ketone ester (1,3 butanediol-acetoacetate diester) supplementation (SD+KE) for 10 weeks. After 10 weeks, anesthesia was induced in an anesthesia chamber with isoflurane gas mixed with air, and the time until anesthetic induction was measured by a blinded observer. Ketone body levels in the blood were measured immediately after induction of anesthesia with a PRECISION XTRA® blood glucose and ketone monitoring kit.

TABLE 1

| Macronutrient | Standard | Ketogenic |
|---|---|---|
| Information | Diet (SD) | Diet (KD) |
| % Cal from Fat | 18.0 | 77.1 |
| % Cal from Protein | 24.0 | 22.4 |
| % Cal from Carbohydrate | 58.0 | 0.5 |
| Caloric Density | 3.1 Kcal/g | 4.7 Kcal/g |

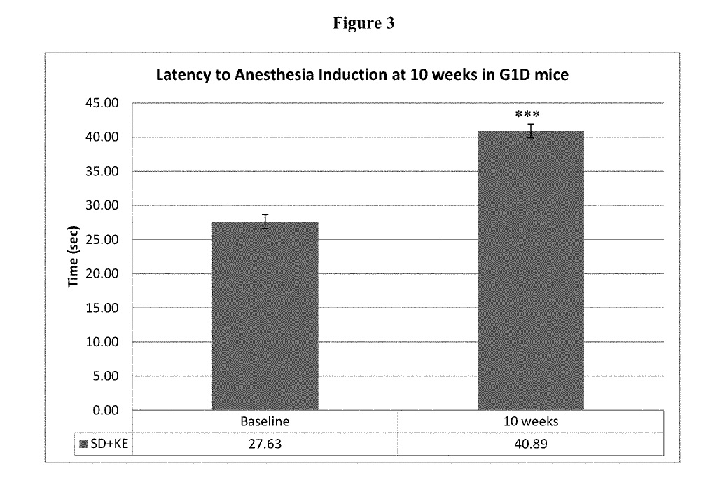

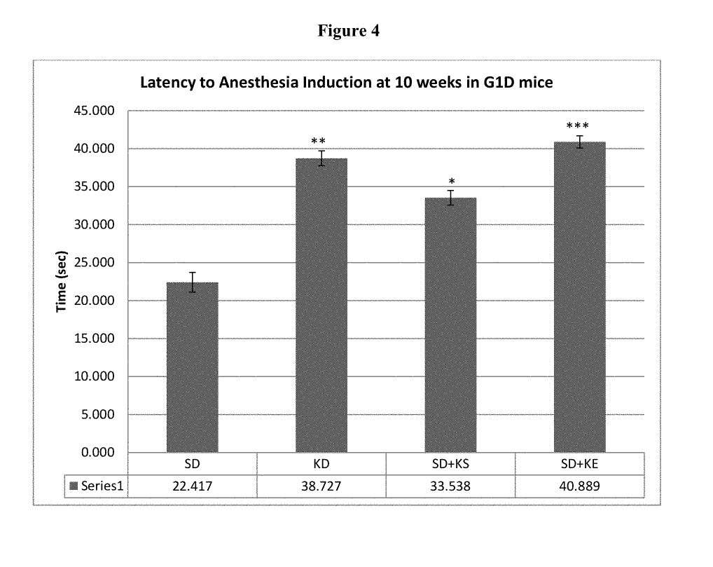

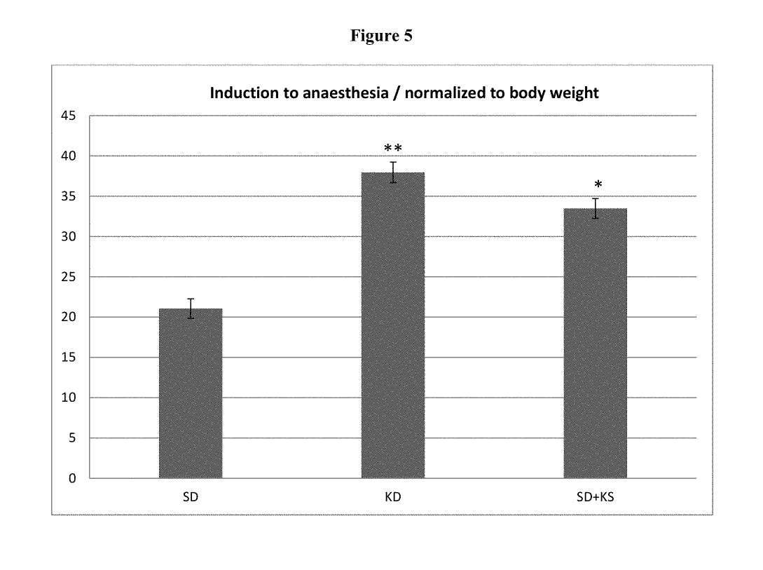

Chronic administration of standard diet with a ketone ester in the G1D mice caused a significant increase in the number of seconds required before anesthetic induction, relative to baseline levels (p=0.0035) (FIG. 3). Mice fed a ketogenic diet, a standard diet with ketone salt, or a standard diet with ketone ester also exhibited significant increases in the time required before anesthetic induction (p=0.0003, p=0.0136, p=0.0003, respectively), relative to control mice fed a standard diet (FIG. 4). The results remained significant, even when the delay in anesthetic induction was normalized to body weight (p=0.0048 for mice fed a ketogenic diet; p=0.0338 for mice fed a standard diet with ketone salt, relative to animals fed a standard diet) (FIG. 5).

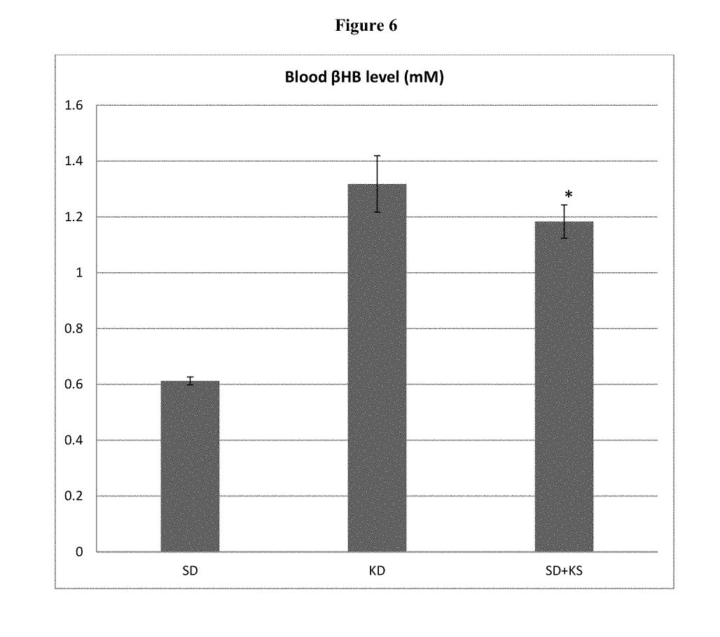

The mice fed a ketogenic diet or a ketone salt supplement also exhibited elevated and sustained blood levels of βHB (p=0.07 for ketogenic diet, p=0.03 for standard diet with ketone salt, relative to control mice fed a standard diet) (FIG. 6), indicating a state of ketosis in these animals.

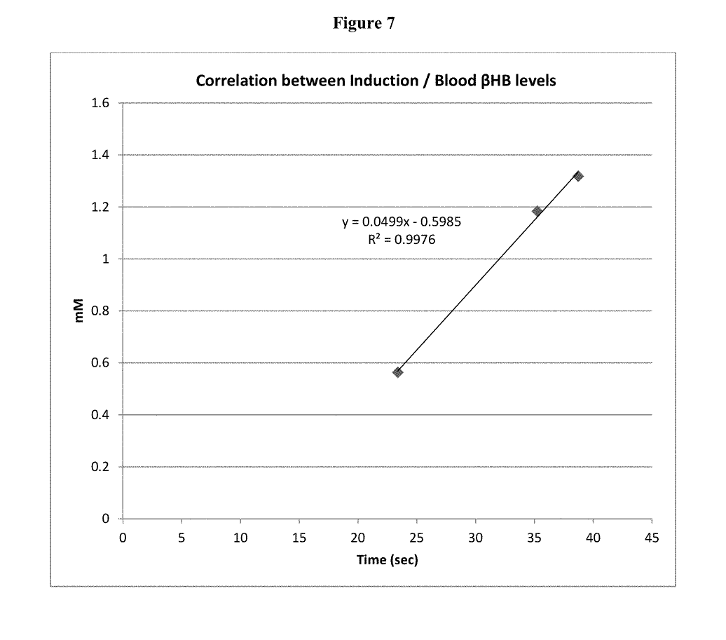

The increase in time required before anesthetic induction also positively correlated with the increase in blood levels of βHB (R=0.9976) (FIG. 7).

Overall, mice given a ketogenic diet or a standard diet with ketone salt supplementation exhibited elevated blood ketone levels and a delay in anesthetic induction. The results of this example demonstrate that nutritional ketosis delays the onset of anesthetic induction.

For reasons of completeness, various aspects of the invention are set out in the following numbered clauses:

Clause 1: A method of delaying the onset of anesthetic induction, comprising administering to a subject a composition comprising one or more ketone supplements, wherein ketosis is induced in the subject.

Clause 2: The method of clause 1, wherein the one or more ketone supplements is at least one of a ketone salt, a ketone ester, or a ketone body precursor, or any combination thereof.

Clause 3: The method of clause 2, wherein the ketone supplement is 1,3-butanediol acetoacetate.

Clause 4: The method of clause 2, wherein the ketone ester is 1,3-butanediol-acetoacetate monoester.

Clause 5: The method of clause 2, wherein the ketone ester is 1,3 butanediol-acetoacetate diester.

Clause 6: The method of clause 2, wherein the ketone salt comprises sodium (Na⁺), potassium (K⁺), or a combination of Na⁺ and K⁺.

Clause 7: The method of clause 2, wherein the ketone salt comprises β-hydroxybutyrate (βHB) mineral salt.

Clause 8: The method of clause 7, wherein the ketone salt comprises a Na⁺/K⁺βHB mineral salt.

Clause 9: The method of clause 2, wherein the ketone salt comprises about 45% to about 55% of a solution.

Clause 10: The method of clause 9, wherein the ketone salt comprises a 50% solution of 375 mg/g of βHB mineral salt per 125 mg/g of the ketone salt.

Clause 11: The method of clause 10, wherein the ketone salt is administered in a dose of 1000-1500 mg.

Clause 12: The method of clause 1, wherein the subject is a vertebrate.

Clause 13: The method of clause 12, wherein the vertebrate is a human.

Clause 14: The method of clause 1, wherein the ketone supplement is delivered chronically.

Clause 15: The method of clause 1, wherein the ketone supplement is delivered sub-chronically.

Clause 16: The method of clause 1, wherein ketosis is induced in the subject if one or more ketone bodies are elevated in a blood sample.

Clause 17: The method of clause 16, wherein the ketone body is βHB.

Clause 18: The method of clause 16, wherein the ketone body is acetoacetate.

Clause 19: The method of clause 16, wherein the ketone body is acetone.

Clause 20: A method of delaying the onset of anesthetic induction, comprising administering a ketogenic diet to a subject, wherein ketosis is induced in the subject.

Clause 21: The method of clause 20, wherein the ketogenic diet comprises fats, proteins, and carbohydrates.

Clause 22: The method of clause 21, wherein the ketogenic diet comprises about 70% to about 80% fat, about 20% to about 25% protein, and about 0.1% to about 1.0% carbohydrate.

Clause 23: The method of clause 22, wherein the ketogenic diet comprises about 77.1% fat, about 22.4% protein, and about 0.5% carbohydrate

Clause 24: The method of clause 23, wherein the ketogenic diet has a caloric density of about 4.7 Kcal/g.

Clause 25: The method of clause 20, wherein the ketogenic diet is administered with a composition comprising a ketone supplement.

Clause 26: The method of clause 25, wherein the ketone supplement is at least one of a ketone salt, a ketone ester, or a ketone body precursor, or any combination thereof.

Clause 27: The method of clause 20, wherein the subject is a vertebrate.

Clause 28: The method of clause 27, wherein the vertebrate is a human.

Clause 29: The method of clause 20, wherein the ketogenic diet is delivered chronically.

Clause 30: The method of clause 20, wherein the ketogenic diet is delivered sub-chronically.

Clause 31: The method of clause 20, wherein ketosis is induced in the subject if one or more ketone bodies are elevated in a blood sample.

Clause 32: The method of clause 31, wherein the ketone body is βHB.

Clause 33: The method of clause 31, wherein the ketone body is acetoacetate.

Clause 34: The method of clause 31, wherein the ketone body is acetone.

What is claimed is:

1. A method of delaying the onset of anesthetic induction, comprising administering to a subject a composition comprising one or more ketone supplements, wherein ketosis is induced in the subject.

2. The method of claim 1, wherein the one or more ketone supplements is at least one of a ketone salt, a ketone ester, or a ketone body precursor, or any combination thereof.

3. The method of claim 2, wherein the ketone supplement is 1,3-butanediol acetoacetate.

4. The method of claim 2, wherein the ketone ester is 1,3-butanediol-acetoacetate monoester.

5. The method of claim 2, wherein the ketone ester is 1,3 butanediol-acetoacetate diester.

6. The method of claim 2, wherein the ketone salt comprises sodium (Na⁺), potassium (K⁺), or a combination of Na⁺ and K⁺.

7. The method of claim 2, wherein the ketone salt comprises β-hydroxybutyrate (βHB) mineral salt.

8. The method of claim 7, wherein the ketone salt comprises a Na⁺/K⁺βHB mineral salt.

9. The method of claim 2, wherein the ketone salt comprises about 45% to about 55% of a solution.

10. The method of claim 9, wherein the ketone salt comprises a 50% solution of 375 mg/g of βHB mineral salt per 125 mg/g of the ketone salt.

11. The method of claim 2, wherein the ketone salt is administered in a dose of 1000-1500 mg.

12. The method of claim 1, wherein the subject is a vertebrate.

13. The method of claim 12, wherein the vertebrate is a human.

14. The method of claim 1, wherein the composition is delivered chronically.

15. The method of claim 1, wherein the composition is delivered sub-chronically.

16. The method of claim 1, wherein ketosis is induced in the subject if one or more ketone bodies are elevated in a blood sample.

17. The method of claim 16, wherein the ketone body is βHB.

18. The method of claim 16, wherein the ketone body is acetoacetate.

19. The method of claim 16, wherein the ketone body is acetone.

20. A method of delaying the onset of anesthetic induction, comprising administering to a subject a ketogenic diet, wherein ketosis is induced in the subject.

21. The method of claim 20, wherein the ketogenic diet comprises fats, proteins, and carbohydrates.

22. The method of claim 21, wherein the ketogenic diet comprises about 70% to about 80% fat, about 20% to about 25% protein, and about 0.1% to about 1.0% carbohydrate.

23. The method of claim 22, wherein the ketogenic diet comprises about 77.1% fat, about 22.4% protein, and about 0.5% carbohydrate

24. The method of claim 23, wherein the ketogenic diet has a caloric density of about 4.7 Kcal/g.

25. The method of claim 20, wherein the ketogenic diet is administered with a composition comprising a ketone supplement.

26. The method of claim 25, wherein the ketone supplement is at least one of a ketone salt, a ketone ester, or a ketone body precursor, or any combination thereof.

27. The method of claim 20, wherein the subject is a vertebrate.

28. The method of claim 27, wherein the vertebrate is a human.

29. The method of claim 20, wherein the ketogenic diet is delivered chronically.

30. The method of claim 20, wherein the ketogenic diet is delivered sub-chronically.

31. The method of claim 20, wherein ketosis is induced in the subject if one or more ketone bodies are elevated in a blood sample.

32. The method of claim 31, wherein the ketone body is βHB.

33. The method of claim 31, wherein the ketone body is acetoacetate.

34. The method of claim 31, wherein the ketone body is acetone.
